(19) United States
(12) Patent Application Publication
(10) Pub. No.: US 2004/0059244 A1
(43) Pub. Date: Mar. 25, 2004

(54) Thermography catheters allowing for rapid exchange and methods of use

(75) Inventors: Jesus Flores, Perris, CA (US); Tracy D. Maahs, Margarita, CA (US); Blair D. Walker, Mission Viejo, CA (US)

Correspondence Address: O'MELVENY & MEYERS, 114 PACIFICA, SUITE 100, IRVINE, CA, 92618, US

(73) Assignee: Volcano Therapeutics, Inc., Laguna Hills, CA

(21) Appl. No.: 10/342,911

(22) Filed: Jan. 14, 2003

Related U.S. Application Data
(63) Continuation of application No. 10/253,391, filed on Sep. 23, 2002.

(51) Int. Cl. A61B 5/00

(52) U.S. Cl. 600/549

(57) ABSTRACT

An intravascular thermography device comprising an elongate catheter having a proximal end, a distal end, a distal guidewire port in a distal region of the catheter, a proximal guidewire port at a location closer to the distal end of the catheter than the proximal end, and a lumen is described. The lumen extends between the proximal guidewire port and the distal guidewire port and is adapted to receive a guidewire. An expansion frame is attached to the catheter at a location distal to the proximal guidewire port. The expansion frame is operable between a contracted condition and an expanded condition, and has at least one temperature sensor. Methods of use are also described.

This is a continuation of U.S. application Ser. No. 10/253,391, filed Sep. 23, 2002, which is incorporated herein by reference in its entirety.

### FIELD OF THE INVENTION

The present invention relates to intravascular thermography devices useful for detection and treatment of vulnerable plaques, and in particular thermography catheters that allow for rapid removal and replacement by an interventional therapeutic catheter. The presence of inflammatory cells within vulnerable plaque and thus the vulnerable plaque itself can, according to the present invention, be identified by detecting heat associated with the metabolic activity of these inflammatory cells.

### BACKGROUND

Cardiovascular disease is one of the leading causes of death worldwide. In the United States each year approximately 1.5 million patients experience a myocardial infarction from atherosclerotic coronary disease. Atherosclerosis is a common form of arteriosclerosis in which deposits of yellowish plaques or atheromas are formed within the intima and inner media of large and medium-sized arteries. These atheromas usually contain cholesterol, lipoid material, and lipophages. The pathological sequence of events leading to acute myocardial infarction includes plaque rupture with exposure of the subintimal surface of the plaque to coronary blood flow. As a result, activation of platelets and the coagulation pathway occurs as the contents of the atherosclerotic plaque interact with circulating blood components. Platelet activation also releases numerous chemical mediators, including thromboxane A2, a vasoconstrictive substance that often leads to localized vasospasm that further impedes coronary artery blood flow. The net result of these events is thrombus formation causing interruption of coronary blood flow to myocardial tissues, causing myocardial necrosis.

According to recent studies, plaque rupture may trigger 60 to 70 percent of fatal myocardial infarction. Plaque erosion or ulceration is the trigger in approximately 25 to 30 percent of fatal infarctions. Unfortunately, vulnerable plaques are often undetectable using conventional techniques such as angiography. The majority of vulnerable plaques that lead to infarction occur in coronary arteries that appeared normal or only mildly stenotic on angiogram performed prior to infarction. Studies on the composition of vulnerable plaque suggest that the presence of inflammatory cells, such as leukocytes and macrophages, is the most powerful predictor of ulceration and/or imminent plaque rupture. For example, in plaque erosion, the endothelium beneath the thrombus is replaced by or interspersed with inflammatory cells.

If vulnerable plaques can be identified, systemic or localized treatments may be performed to prevent development of acute coronary syndromes. These treatments include inserting a catheter into the coronary artery to remove or remodel the plaque using atherectomy or balloon angioplasty. Localized or light activated drug, or localized thermal, cryogenic, ultrasound or radiation therapy may be delivered to combat inflammation. At the present time, when more than one interventional device, such as a thermography catheter, an angioplasty catheter, a stent deployment catheter, and an atherectomy catheter, are used during a procedure, exchange of one catheter for another occurs frequently and becomes problematic. The process of introducing the second catheter may require the use of an “exchange length” navigating wire that can be as long as 300 centimeters in length. The wire can be quite awkward to use, requiring two individuals to assure that the wire does not engage in erratic movements or exit the sterile area of the operation. In addition, manipulating a standard length guidewire (175-190 cm) also can require two operators when the thru-lumen of the catheter extends its entire length (140-150 cm), as for an over-the-wire catheter. Operating such a wire may also increase the procedural time because the operators need to coordinate their manipulation of the catheter and wire to prevent accidental movement of a device that is intended to remain stationary during this exchange.

Devices and methods are therefore needed to provide accurate detection, treatment, and/or removal of vulnerable plaque in blood vessels, especially in the coronary arteries, and to allow for rapid removal and replacement of working or therapeutic devices by a single operator.

### SUMMARY OF THE INVENTION

The present invention provides intravascular thermography devices useful for detection and treatment of vulnerable plaques. The presence of inflammatory cells within vulnerable plaque and thus the vulnerable plaque itself can, according to the present invention, be identified by detecting heat associated with the metabolic activity of these inflammatory cells. Specifically, activated inflammatory cells have a heat signature that is slightly above that of connective tissue cells. Accordingly, one can determine whether a specific plaque is vulnerable to rupture and/or ulceration by measuring the temperature of the arterial wall in the region of the plaque. Thermography catheters that are capable of thermally mapping blood vessels to identify thermal hot spots are described in Campbell et al., U.S. Pat. No. 6,245,026, Brown, U.S. Pat. No. 5,871,449, Cassells et al., U.S. Pat. No. 5,935,075, and Campbell, U.S. Pat. No. 5,924,997, each of which are incorporated herein by reference.

The devices of the present invention, however, do not require usage of the conventional “exchange length” guidewire, thereby allowing rapid exchange (by a single operator) with other interventional devices, such as an angioplasty catheter, stent deployment catheter, or an atherectomy catheter. In certain embodiments, the device includes an elongate catheter having a proximal end, a distal end, a distal guidewire port in the distal end of the catheter, a proximal guidewire port at a location closer to the distal end of the catheter than the proximal end, and a lumen shaped to slideably receive a guidewire. The guidewire lumen extends between the proximal guidewire port and the distal guidewire port.

An expansion frame is attached to the catheter at a location distal to the proximal guidewire port. The expansion frame is contained in a contracted or low profile condition that facilitates movement through tortuous vessels so that its can be positioned within a region of interest in a coronary artery. The frame is thereafter expanded, and may achieve contact with the endoluminal surface of the vessel in certain embodiments. The expansion frame carries at least one temperature sensor, e.g., a thermocouple or a thermistor. Each temperature sensor carried by the expansion frame is connected to wires extending to the proximal end of the thermography device so that temperature readings may be recorded after deployment of the expansion frame. In certain embodiments, the expansion frame consists of a plurality of flexible struts that, when deployed, bow radially outward. The frame may include three struts, four struts, five struts, six struts, or any other suitable number of struts. In other embodiments, each strut carries a temperature sensor.

A capture sheath is slideably disposed around the expansion frame and contains the expansion frame in its low-profile condition. The capture sheath is operated from the proximal end of the catheter to slide either proximally or distally and thereby release the expansion frame. The capture sheath has a slotted aperture in its distal region. The slot aligns with the proximal guidewire port of the catheter and allows passage of the guidewire from the guidewire lumen of the catheter to the outside surface of the capture sheath. The slot, typically longitudinally elongated, allows the capture sheath to slide relative the inner catheter and still accommodate passage of the guidewire.

A registry mechanism is provided to maintain circumferential alignment between the proximal guidewire port of the catheter and the slot in the distal region of the capture sheath. The registry mechanism in certain embodiments consists of the complimentary fit between the catheter and the capture sheath where the catheter and the capture sheath have an oval or elliptical cross-section. In other embodiments, the registry mechanism comprises a complimentary fit between a longitudinal rib on the outer surface of the catheter and a longitudinal groove on the inner surface of the capture sheath.

Where the expansion frame comprises a plurality of struts, the struts may be formed of a self-expanding material, in certain cases a shape memory alloy or a shape memory polymer. In other embodiments, the material will be superelastic, e.g., nitinol. Shape memory alloys are desirable because of their ability to be processed and “shape set” into a desired final configuration, then manipulated into a low profile configuration that may be more easily navigated through a torturous location in the body, such as a coronary artery. This shape setting is typically achieved by heating the shape memory alloys above a certain temperature known as the “transition temperature,” which causes any deformation introduced below the transition temperature to be reversed.

Additionally, the use of stress-induced martensite alloys decreases the temperature sensitivity of the devices, making them easier to navigate and deploy. The use of these alloys are discussed in detail in Krumme, U.S. Pat. No. 4,485,816, and Jervis, U.S. Pat. Nos. 4,665,906 and 6,306,141, each of which are incorporated herein by reference.

Shape memory polymers can be shape set in seconds at around 70° C., and can withstand deformations of several hundred percent. For example, oligo(e-caprolactone) dimethacrylate incorporates a crystallizable transitioning segment that determines both temporary and permanent shape of the polymer. By manipulating the quantity of co-monomer, n-butyl acrylate, in the polymer, the cross-link density can be adjusted, thereby allowing one to vary mechanical strength and transition temperature over a side area, depending on the needs of a particular device. Homo-polymers of both monomers are known to be biocompatible. In addition, binary alloys such as tantalum-tungsten and tantalum-niobium have been used in the manufacture of medical devices such as stents and other supportive structures as a means of enhancing their radiopacity. This enhanced radiopacity allows for better visual tracking, and increases the accuracy of device placement when used in conjunction with fluoroscopy and quantitative coronary angiography. The use of binary alloys is discussed in detail in Pacetti et al., WO02/05863, which is incorporated herein by reference.

The thermography device of the present invention may also be equipped with capabilities for flushing blood from an annulus between the catheter and the capture sheath. For example, where flushing is to occur down the central lumen of the catheter, the guidewire lumen of the catheter may extend and communicate with the proximal end of the catheter. In this case, the lumen terminates proximally in a flushing port, typically having a luer adaptor to receive flushing solution. The proximal port typically includes a valve to prevent blood loss when flushing is not performed, for example, a one-way valve, a pressure-activated valve, or a luer-activated valve. Flushing ports in a distal region of the catheter allow fluid to pass into the annulus between the catheter and the capture sheath and a seal will prevent the fluid from flowing proximally within the annulus. On the other hand, where flushing is to occur down the annulus between the catheter and the capture sheath, the annulus will extend and communicate with the proximal end of the catheter. Ports and valves, as noted above, are provided to inject flushing solution into the annulus.

In use, the interventional cardiologist introduces a first guidewire (such as an 0.035″ guidewire for guiding catheter introduction) into a peripheral artery and advances the first guidewire and guiding catheter to the aortic arch. The first guidewire is pulled back, allowing the guiding catheter to position in the coronary ostium. The first guidewire is removed. A second guidewire (such as a 0.014″ coronary guidewire) is then advanced to a position across a region of interest within a target vessel. Typically the devices are introduced into a femoral artery, brachial artery, axillary artery, or a subclavian artery. The region of interest is generally within a coronary artery having a vulnerable plaque, generally the left anterior descending coronary artery, the left circumflex coronary artery, the right coronary artery, the left obtuse marginal artery, the left diagonal arteries, and the posterior descending artery. The region of interest may alternatively be within an artery of the head and neck, i.e., an artery that supplies blood to the head, including the common carotid artery, the internal carotid artery, the middle cerebral artery, the anterior cerebral artery, the posterior cerebral artery, the vertebral artery, and the basilar artery.

A guiding catheter is advanced over the first guidewire and positioned to facilitate entry into the artery of interest, e.g., into the coronary ostium where a coronary artery is to be studied. After removal of the first guidewire, the proximal end of the second guidewire is inserted into the distal guidewire port of the catheter and is advanced through the guidewire lumen, through the proximal guidewire port, and through the slot in the distal region of the capture sheath. The capture sheath covers the expansion frame. The catheter and capture sheath are then advanced as an assembly along the guidewire until the expansion frame is located within the region of interest. The capture sheath is slid proximally or distally to release the expansion frame. Alternatively, the capture sheath could be held in place, and the catheter advanced out of the capture sheath to release the expansion frame. The expansion frame and the temperature sensors expand, and preferably contact the endoluminal surface of the vessel. The temperature sensors then measure the temperature of the endoluminal surface of the vessel. This temperature reading is then compared with temperature readings taken at different locations along the endoluminal surface, and/or a temperature reading of blood within the vessel. An elevated temperature reading at the region of interest will indicate a likelihood of having vulnerable plaques.

After thermography, the capture sheath is slid into a position covering the expansion frame, thereby regaining a low-profile configuration. The catheter and the capture sheath are then withdrawn over the guidewire and removed from the patient. It will be understood that the thermography catheter can be exchanged for an interventional procedural catheter with minimal guidewire length extending from the patient. This fact is due to the ability of the catheter to track over the guidewire for only a relatively short distance at the distal end of the catheter. The proximal guidewire port is located closer to the distal end of the catheter than the proximal end, and will typically be located 10 centimeter or more from the distal end of the catheter, 15 centimeters or more from the distal end of the catheter, 20 centimeters or more from the distal end of the catheter, 25 centimeters or more from the distal end of the catheter, 30 centimeters or more from the distal end of the catheter, but in any case the proximal guidewire port will be closer to the distal end of the catheter than the proximal end of the catheter.

It is typically desirable to have the proximal guidewire port located at a position where the guidewire will emerge from both the catheter and the capture sheath but remain within the guiding catheter so that the guidewire is not exposed to the vascular endothelium in order to prevent injury to the vessel wall. It may also be desirable to have the proximal guidewire port located at a position within the guiding catheter that is relatively straight, i.e., it is desirable to avoid having the proximal guidewire port located at a position within the highly curved region of the curved region of “the guiding catheter shape,” and it may even be desirable to avoid having the proximal guidewire port located within the guiding catheter in the moderately curved aortic arch. Where the proximal guidewire port is located at a position within the guiding catheter in a highly curved anatomy, it may be difficult for the catheter to track smoothly over the guidewire.

After the thermography catheter is removed, the cardiologist can insert over the guidewire an angioplasty catheter, a stent placement catheter, an atherectomy catheter, or catheters for localized thermal, cryogenic, radiation, or ultrasonic therapy to stabilize or remove vulnerable plaques. After treatment of the vulnerable plaques, the interventional therapeutic catheter is removed.

In another embodiment, the thermography catheter includes an inner assembly that nests within an outer assembly. The inner assembly comprises an elongate member that is a mandrel or a tubular mandrel. An expansion frame is coupled to the distal end of the elongate member. The expansion frame will carry at least one temperature sensor and typically a plurality of temperature sensors, for example, three temperature sensors, four temperature sensors, five temperature sensors, six temperature sensors, or any other suitable number of temperature sensors. The expansion frame operates to expand from a low-profile contracted condition suitable for navigating tortuous vessels, to an expanded condition that preferably achieves contact with the endoluminal surface at the region of interest. The inner assembly further includes a first tubular member bonded adjacent the distal end of the elongate member, the first tubular member adapted to receive and slide over a guidewire.

The outer assembly comprises an elongate tubular member having a proximal end, a distal end, and a lumen therebetween. A second tubular member is bonded adjacent the distal end of the elongate tubular member. A capture sheath is coupled to the distal end of the elongate tubular member and extends distally thereof. The thermography catheter is assembled by sliding the inner assembly within the outer assembly so that the expansion frame is covered by the capture sheath, the elongate member of the inner assembly fits within the elongate tubular member, and the first tubular member of the inner assembly fits within the second tubular member of the outer assembly. In certain embodiments, the expansion frame is carried at the distal end of the elongate member of the inner assembly. In other embodiments, the expansion frame is bonded to a third tubular member that is coupled in turn to the distal end of the elongate member of the inner assembly. As with the thermography catheter of other embodiments described above, here the expansion frame may be formed of a plurality of flexible struts that bow radially outward, and the struts may be a shape-memory alloy or polymer, or a superelastic material, e.g., nitinol.

The lumen of the elongate tubular member of the outer assembly may communicate with a flushing port at a proximal end of the thermography catheter. In this case, the lumen is adapted to receive a solution for flushing blood from the annulus between the capture sheath and the expansion frame, the annulus between the first tubular member of the inner assembly and the second tubular member of the outer assembly, and the annulus between the elongate tubular member of the outer assembly and the elongate member of the inner assembly. In certain cases, the elongate member of the inner assembly is a tubular mandrel or tubular member. In this case, the lumen of the tubular member of the inner assembly may communicate with a flushing port at the proximal end and one or more ports at the distal end of the thermography catheter. This lumen receives fluid for flushing blood from the annulus between the first tubular member of the inner assembly and the second tubular member of the outer assembly, the annulus between the capture sheath and the expansion frame, and the annulus between the elongate tubular member of the outer assembly and the elongate member of the inner assembly. Where flushing capabilities are present, the flushing port at the proximal end of the thermography catheter includes a valve to prevent blood loss when flushing is not performed, and to prevent bleed-back proximally into the catheter and annulus, which might inhibit smooth movement of sliding components. The valve can be any of a one-way valve, a pressure-activated valve, and a luer-activated valve.

The flushing port at the proximal end of the thermography catheter may include, in addition to the aforementioned valve, a fluid chamber having a dynamic seal that permits relative axial movement between the two assemblies without loss of fluid. In certain cases the slider moves proximal to withdraw the capture sheath to release the expansion frame. In other cases, the injection tube slides forward to advance the expansion frame beyond the capture sheath. The fluid chamber is defined by a support tube that contains the point of fluid entry (i.e., the valve), a tubular slider that is bonded to a proximal region of the outer assembly, and a dynamic seal between the support tube and the tubular slider. In this arrangement, the lumen of the elongate tubular member of the outer assembly communicates with the fluid chamber and allows sliding of the outer assembly relative to the inner assembly without loss of fluid. When the lumen of the tubular member of the inner assembly is used for flushing, the tubular member advantageously includes an annular seal to provide fluid resistance, and preferably to prevent fluid from escaping proximally through the lumen of the elongate tubular member of the outer assembly.

Each temperature sensor includes wires extending to the proximal end of a catheter to record temperature readings at the region of interest. In certain embodiments, the temperature sensor wires extend proximally within the lumen of the tubular member of the inner assembly. In other embodiments, the temperature sensor wires extend proximally within the elongate tubular member of the outer assembly.

The elongate tubular member of the outer assembly may be formed of hypo tube. It may be desirable to construct the thermography catheter so that the distal end of the catheter is more flexible than the proximal end of the catheter. Moreover, a gradual transition between these two sections is desired to avoid kinking and to maximize advancing capabilities. This can be accomplished by creating a flexible transition region on the distal section of the elongate member of the inner or outer assembly, e.g., a spiral cut hypo tube, a laser-welded spring, a tapered mandrel bonded to the distal end of a tubular elongate member of the inner or outer assembly, or a tapered mandrel where the mandrel is the elongate member of the inner assembly.

The methods of use of this thermography catheter will be understood to be similar to the methods described above. A guidewire is positioned across a region of interest within a target vessel. The proximal end of the guidewire is inserted into the first tubular member of the inner assembly. The catheter is advanced along the guidewire until the temperature sensors are located within the region of interest. The capture sheath is slid proximally or distally to release the temperature sensors. The temperature sensors are operated to measure the temperature of an endoluminal surface of the vessel.

### BRIEF DESCRIPTION OF THE DRAWINGS

FIG. 1A depicts a thermography catheter according to the present invention having a slotted capture sheath slid proximally to release a strutted temperature sensor assembly.

FIG. 1B depicts the thermography catheter of FIG. 1A with the capture sheath disposed about the strutted temperature sensor assembly.

FIG. 2A depicts a guidewire and guiding catheter disposed within a region of interest within a blood vessel.

FIG. 2B depicts a thermography catheter according to the present invention advanced over the guidewire and disposed within the region of interest.

FIG. 2C depicts the thermography catheter of FIG. 2B measuring temperature of a plaque after release of the expansion frame.

FIG. 2D depicts the removal of the thermography catheter from the region of interest after collapse of the expansion frame by the capture sheath.

FIG. 2E depicts angioplasty at the region of interest after exchange of an angioplasty catheter for the thermography catheter.

FIG. 2F depicts stent deployment at the region of interest after exchange of a stent-placement catheter for the thermography catheter.

FIG. 2G depicts the removal of the guidewire after removal of the stent-placement catheter.

FIG. 2H depicts the stent at the region of interest after removal of the guiding catheter.

FIG. 3A depicts a thermography catheter having a slotted capture sheath, and wherein the catheter and the capture sheath have an oval cross-section that provides a complimentary fit between the catheter and the capture sheath.

FIG. 3B depicts a cross-sectional view of the thermography catheter of FIG. 3A taken through section line B-B.

FIG. 3C depicts the catheter of FIG. 3A having a circular guidewire lumen.

FIG. 3D depicts a cross-sectional view of the thermography catheter of FIG. 3C taken through section line D-D.

FIG. 3E depicts the catheter of FIG. 3A having a circular outer diameter and circular guidewire lumen.

FIG. 3F depicts a cross-sectional view of the thermography catheter of FIG. 3E taken through section line F-F.

FIG. 3G depicts a catheter and capture sheath having a square-geometry complementary fit.

FIG. 3H depicts a catheter and capture sheath having a triangular-geometry complementary fit.

FIG. 4A depicts a cross-sectional view of the thermography catheter and capture sheath, wherein the catheter includes a longitudinal rib and the capture sheath includes a longitudinal groove.

FIG. 4B depicts a cross-sectional view of the thermography catheter and capture sheath, wherein the catheter includes a longitudinal rib and the capture sheath includes a longitudinal groove.

FIG. 4C depicts a cross-sectional view of the thermography catheter and capture sheath, wherein the catheter includes a pair of longitudinal ribs and the capture sheath includes a pair of longitudinal grooves.

FIG. 4D depicts a cross-sectional view of the thermography catheter and capture sheath, wherein the catheter includes a pair of longitudinal ribs and the capture sheath includes a pair of longitudinal grooves.

FIG. 5A depicts an inner assembly of a thermography catheter.

FIG. 5B depicts an outer assembly of a thermography catheter.

FIG. 5C depicts the inner assembly of a thermography catheter nested within the outer assembly.

FIG. 6 depicts the proximal end of the outer assembly with luer adaptor and a dynamic seal for flushing.

FIG. 6A depicts a longitudinal cross-section of the proximal end of the outer assembly of FIG. 6 taken through section line A-A.

FIG. 7A depicts a cross-sectional view through the elongate tubular member of the outer assembly and the mandrel of the inner assembly showing temperature sensor wires disposed alongside the mandrel and within the elongate tubular member.

FIG. 7B depicts cross-sectional view through the elongate tubular member of the outer assembly and the tubular mandrel of the inner assembly showing temperature sensor wires carried within the tubular mandrel.

FIG. 7C depicts cross-sectional view through the elongate tubular member of the outer assembly and the tubular mandrel of the inner assembly showing temperature sensor wires alongside the tubular mandrel and within the elongate tubular member.

FIG. 7D depicts cross-sectional view through the elongate tubular member of the outer assembly and the tubular mandrel of the inner assembly showing temperature sensor wires carried within the tubular mandrel.

FIG. 8A depicts a region of a thermography catheter having a tubular mandrel for flushing and a toroidal seal to prevent escape of fluid proximally.

FIG. 8B depicts a region of a thermography catheter having a matched diameter between the inner assembly and the outer assembly to prevent escape of fluid proximally.

### DETAILED DESCRIPTION

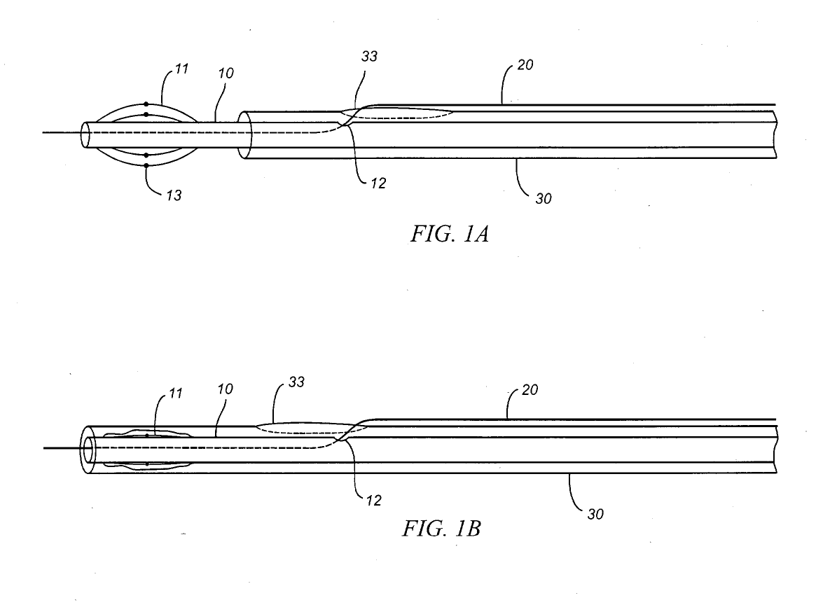

In a first embodiment, a thermography catheter is provided as shown in FIG. 1A. Catheter 10 carries expansion frame 11 at the distal end of catheter 10. Expansion frame 11 comprises of a plurality of struts that bow radially outward when released. Each strut carries temperature sensor 13 at a position on the strut, preferably at the point of maximum expansion. Catheter 10 includes orifice 12 that allows passage of guidewire 20 from the lumen of catheter 10 to a position outside the lumen of catheter 10. Orifice 12 will generally be located closer to the distal end of catheter 10 than the proximal end of the catheter, and will generally be 10 centimeters or more proximal the distal end of catheter 10, and more preferably 20 centimeters or more proximal the distal end of catheter 10. Capture sheath 30 is slideably disposed about catheter 10. Capture sheath 30 includes slotted aperture 33 to allow passage of guidewire 20 to an area outside of capture sheath 30. It is desirable to maintain alignment between orifice 12 and slotted aperture 33 in order to maintain a clear passage for guidewire 20.

When capture sheath 30 slides distally, it compresses and covers expansion frame 11 to provide a low-profile configuration for passage through tortuous vessels. Slotted aperture 33 allows for sliding of capture sheath 30 proximally (to release expansion frame 11) and distally (to compress expansion frame 11), and at all times maintains a clear passage for guidewire 20. By using such an assembly that tracks over guidewire 20 for only a distal portion of the catheter, the thermography catheter can be exchanged for an interventional therapeutic catheter with only a minimal length guidewire outside of the patient's body, and the exchange can be performed by a single operator.

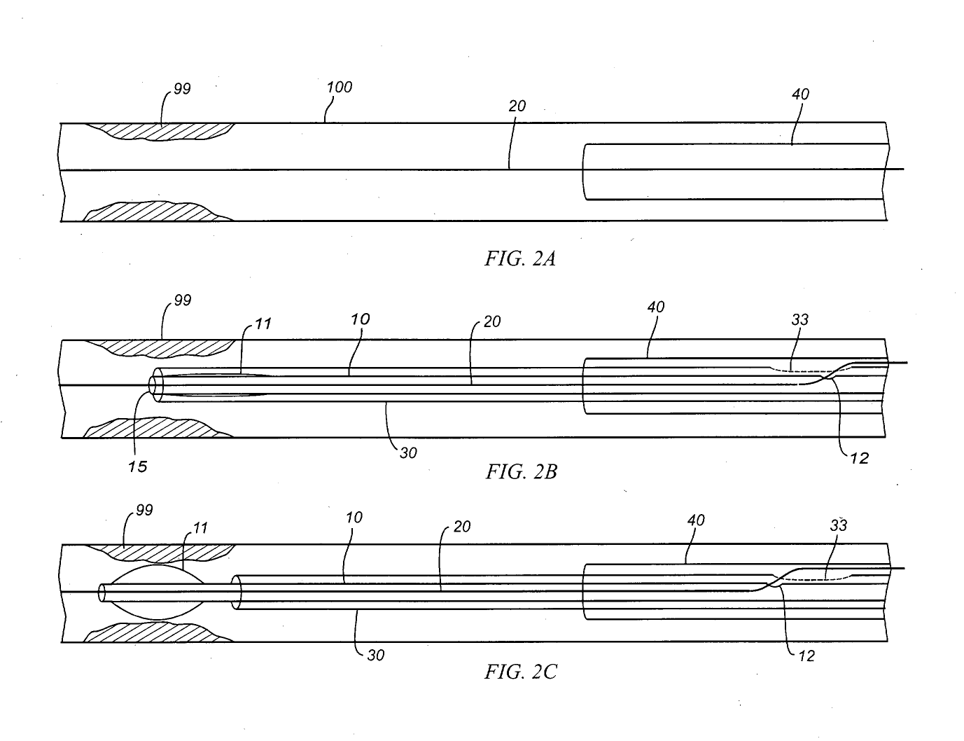

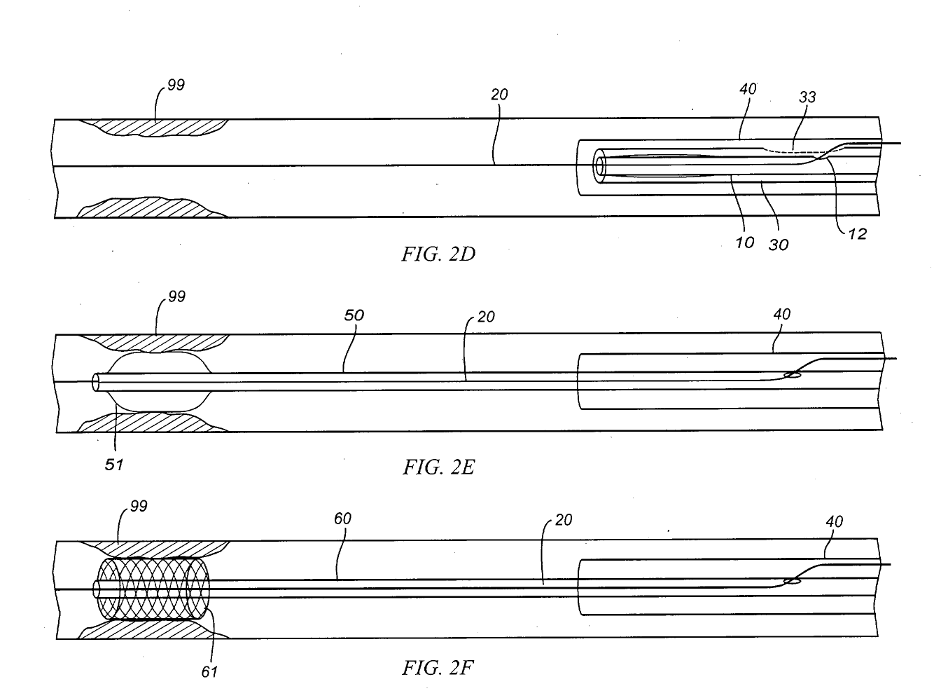

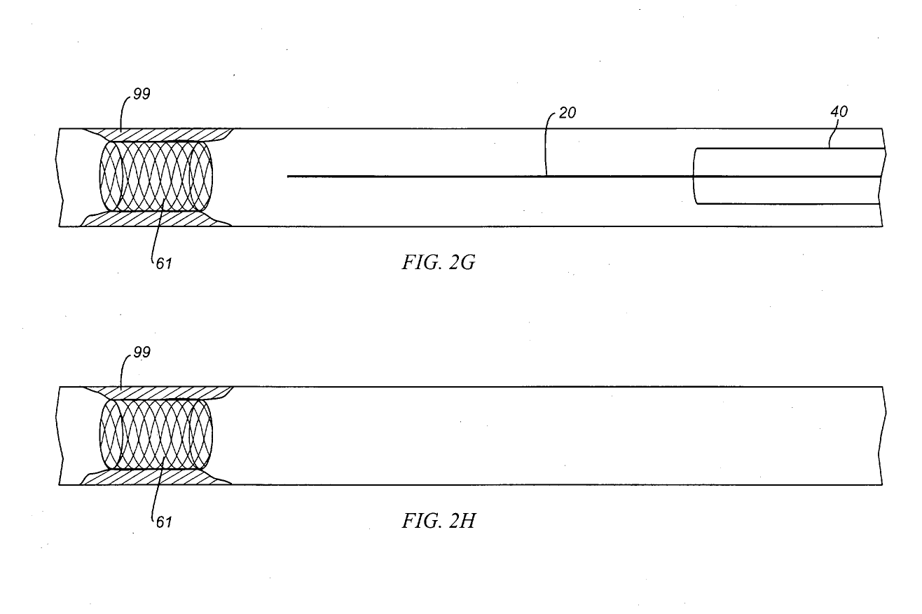

Although the present thermography catheter may initially find use in coronary vessels, it can be used in any vessels where thermographic measurements are desired. Vessel 100 having vulnerable plaque 99 is depicted in FIG. 2A. Coronary guidewire 20 is first introduced through a peripheral artery, such as the femoral artery, the subclavian artery, the brachial artery, or a carotid artery, and advanced to the region of interest and beyond vulnerable plaque 99. The thermography catheter is advanced over guidewire 20 to the region of interest as shown in FIG. 2B. Guidewire 20 passes through distal guidewire port 15 of catheter 10 and exits catheter 10 proximally through orifice 12. Guidewire 20 passes through slotted aperture 33 of capture sheath 30, but preferably is maintained within guiding catheter 40. Expansion frame 11 is positioned adjacent vulnerable plaque 99. Capture sheath 30 is then withdrawn proximally to release expansion frame 11 as shown in FIG. 2C.

Thermographic measurements are taken from the endoluminal surface of plaque 99. Expansion frame 11 is then collapsed by distally advancing capture sheath 30. Thermography catheter 10 and capture sheath 30 are then removed from the region of interest as shown in FIG. 2D. Following removal of the thermography catheter from the proximal end of guidewire 20, an interventional therapeutic procedure can be performed as shown in FIGS. 2E and 2F. Angioplasty catheter 50 is advanced over guidewire 20 as shown in FIG. 2E. After balloon 51 is aligned adjacent plaque 99, angioplasty is performed to compress plaque 99. Alternatively, stent-placement catheter 60 can be advanced over guidewire 20 as shown in FIG. 2F. Stent 61 is deployed to compress plaque 99 after the stent is properly positioned within the region of the vulnerable plaque. In certain embodiments, the stent will incorporate a drug for treating the vulnerable plaque. Stent-placement catheter 60 is then removed, and guidewire 20 is then withdrawn as shown in FIG. 2G. Guiding catheter 40 is then removed, leaving stent 61 as shown in FIG. 2H. Other alternative treatments of vulnerable plaque may include delivering localized or light-activated drugs, or localized thermal, cryogenic, ultrasonic, or radiation therapy to combat inflammation.

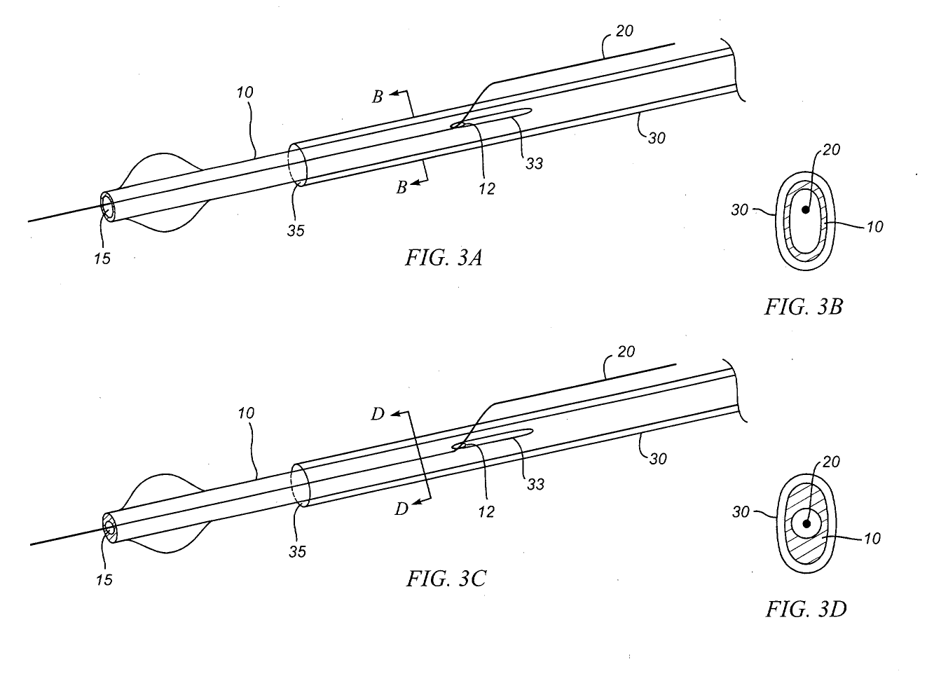

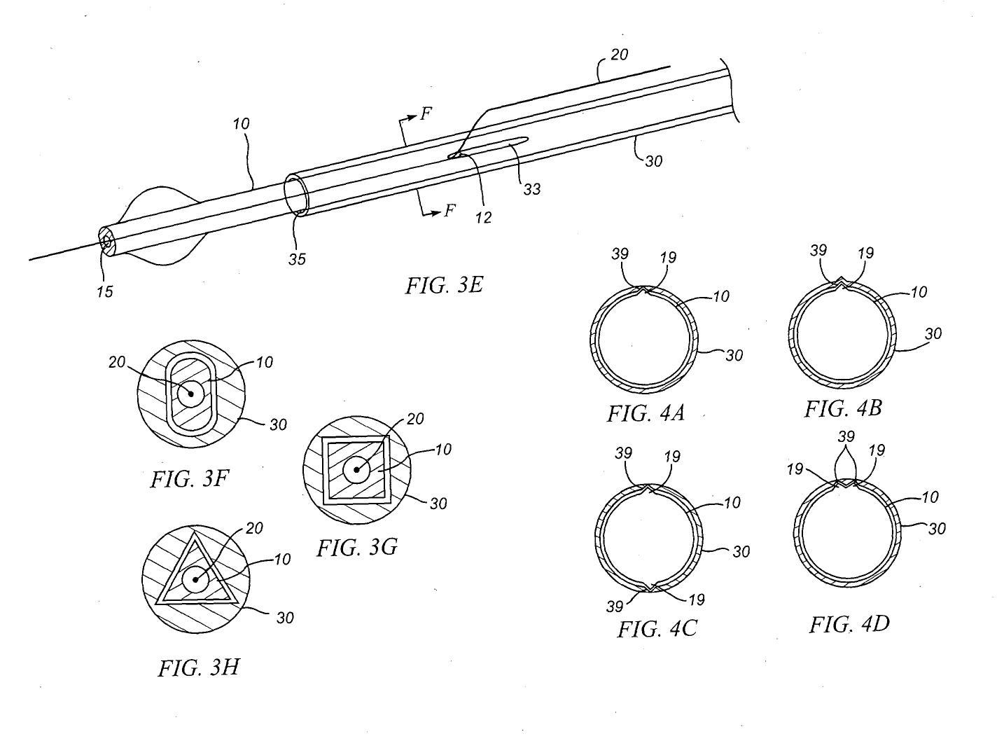

In certain embodiments it is desirable to maintain circumferential alignment between slotted aperture 33 and orifice 12 in order to maintain a clear passage for guidewire 20. To this end, catheter 10 and capture sheath 30 may be constructed with an oval or an elliptical cross-section as shown in FIG. 3A. FIG. 3B shows a cross-sectional view of the assembly of FIG. 3A taken through section line B-B. When catheter 10 is nested within sheath 30, the complementary geometry provides a registry mechanism to maintain circumferential alignment between orifice 12 and slotted aperture 33 of capture sheath 30. Guidewire lumen 15 may have an elliptical geometry as shown in FIG. 3A or circular geometry as shown in FIG. 3C. FIG. 3D shows a cross-sectional view of the assembly of FIG. 3C taken through section line D-D. In other embodiments, the outer diameter of the capture sheath is circular while an elliptical registry mechanism is present, as shown in FIG. 3E, and in cross-section 3F.

Alternative mechanisms for circumferential registry are depicted in FIGS. 3G and 3H, and in FIGS. 4A, 4B, 4C, and 4D. In FIG. 3G a registry mechanism based on square geometry is used. In FIG. 3H a registry mechanism based on triangular geometry is used. In FIG. 4A, catheter 10 includes longitudinal rib 19 shaped to fit within groove 39 formed within the inner surface of capture sheath 30. The outer diameter of capture sheath 30 maintains a smooth cylindrical geometry. In FIG. 4B, catheter 10 includes longitudinal rib 19 shaped to fit within groove 39. Here, both the inner diameter and outer diameter of sheath 30 are formed with groove 39. In FIG. 4C, a pair of longitudinal ribs 19 in catheter 10 and longitudinal grooves 39 in sheath 30 are placed approximately 180° apart. In FIG. 4D, a pair of longitudinal ribs 19 in catheter 10 and longitudinal grooves 39 in sheath 30 are placed adjacent to each other.

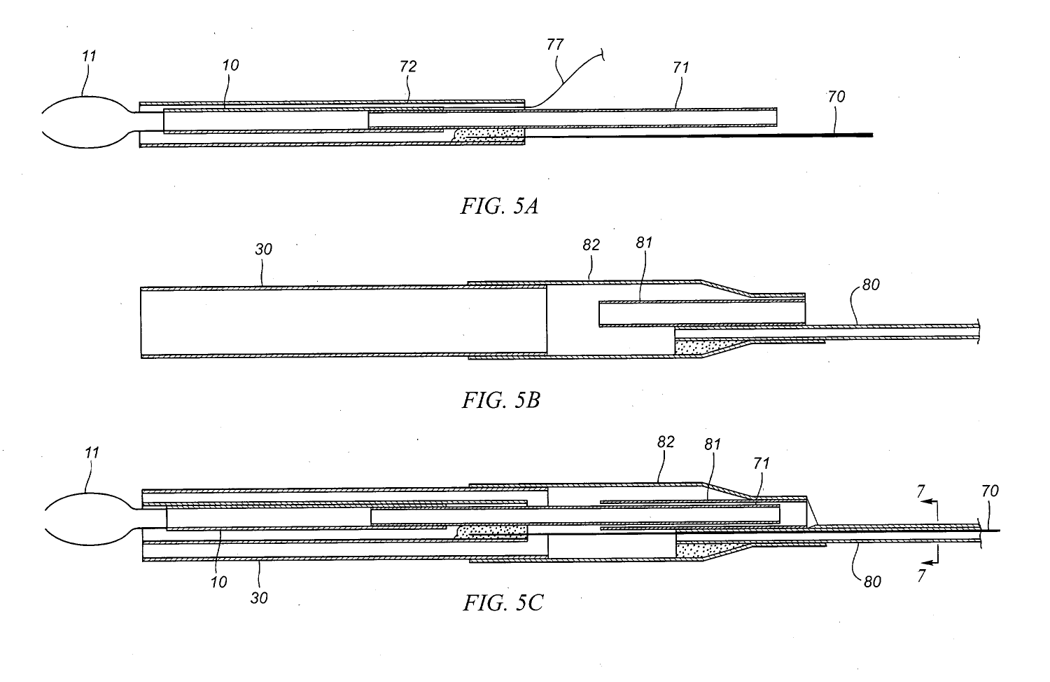

In another embodiment, a thermography catheter having an inner assembly that fits within an outer assembly is shown in FIGS. 5A, 5B, and 5C. The inner assembly comprises elongate member 70, e.g., a mandrel, as shown in FIG. 5A. A first tubular member 71 is bonded adjacent the distal end of mandrel 70. Tubular member 71 includes a lumen adapted to slideably receive a guidewire. Expansion frame 11, having at least one temperature sensor and being operable between a contracted condition and an expanded condition, is bonded to a distal end of catheter 10. Second tubular member 72 is disposed about the distal end of catheter 10, but terminates proximal expansion frame 11.

The outer assembly comprises elongate tubular member 80 having a lumen that extends from the proximal end to the distal end of tubular member 80 as shown in FIG. 5B. Second tubular member 81 is bonded adjacent the distal end of tubular member 80. Capture sheath 30 is bonded distally to transition tubing 82. Tubular member 81 is shaped to receive tubular member 71 of the inner assembly.

The thermography catheter is assembled by nesting the inner assembly within the outer assembly as shown in FIG. 5C. Mandrel 70 is slideably received within tubular member 80 while tubular member 71 is slideably received within tubular member 81. A guidewire is slideably received through the distal end of expansion frame 11 and passes proximally through the lumen of tubular member 71 and tubular member 81. It will be understood that the configuration described above ensures that a clear passage will be maintained at all times for the guidewire to emerge proximally from the guidewire lumen of the inner assembly and the capture sheath. Stated differently, the assembly shown in FIG. 5C will resist rotation of the inner assembly relative to the outer assembly and thereby prevent obstruction of the guidewire passageway.

The methods of use of this thermography catheter will be understood to be similar to the methods described above in FIGS. 2A to 2H. A guidewire is positioned across a region of interest within a target vessel. The proximal end of the guidewire is inserted into tubular member 71 of the inner assembly. The catheter is advanced along the guidewire until the temperature sensors are located within the region of interest. Capture sheath 30 is slid proximally to release the temperature sensors and expansion frame. The temperature sensors are operated to measure the temperature of the endoluminal surface of the vessel at the site of vulnerable plaque 99. The thermography catheter is removed after closing the expansion frame with the capture sheath. Interventional therapeutic catheters as discussed above are then exchanged for the thermography catheter and advanced over the guidewire to treat vulnerable plaque 99.

In certain cases it will be desirable to flush blood from the annulus between tubular member 80 and mandrel 70, the annulus between tubular member 71 of the inner assembly and tubular member 81 of the outer assembly, and the annulus between capture sheath 30 and expansion frame 11. Flushing can be used to avoid penetration of blood between sliding members of the inner and outer assemblies. Penetration of blood is undesirable because blood may clot between the sliding members of the inner and outer assemblies. Even in the absence of clotting, blood will inhibit proper movement due to the higher viscosity of blood.

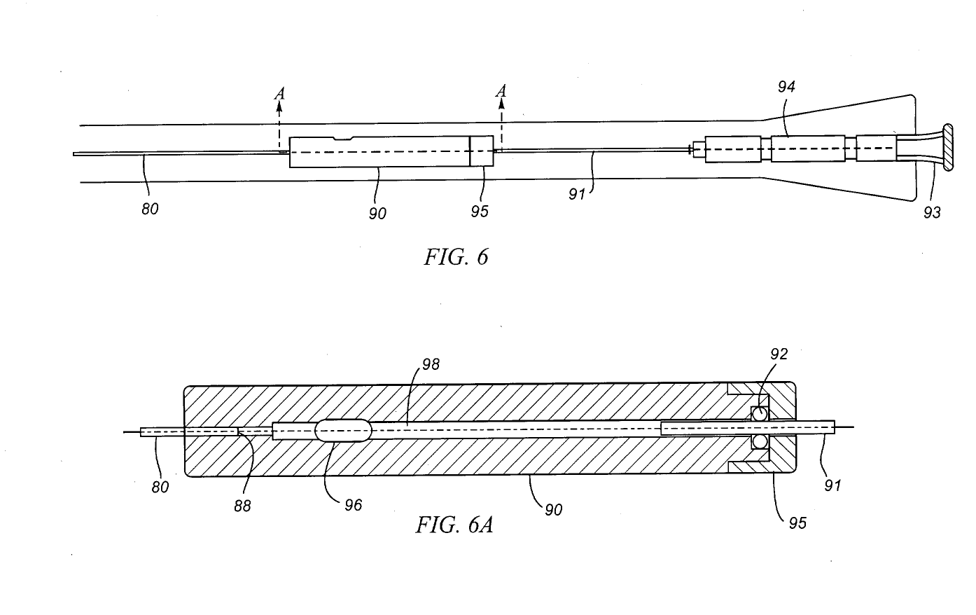

In order to perform flushing the lumen of tubular member 80 of the outer assembly communicates with a flushing port at the proximal end of the thermography catheter as shown in FIGS. 6 and 6A. Tubular member 80 of the outer assembly is bonded proximally to slider body 90, and terminates proximally at flushing port 88. Port 88 communicates with chamber 98 and receives fluid, such as saline, lactated Ringers, or water, for flushing. Chamber 98 is defined by slider body 90 and communicates proximally with injection tube 91. Slider body 90 includes radial hole 96 for a knob. Dynamic seal 92, e.g., an 0-ring, is disposed between slider body 90 and injection tube 91 to enable relative longitudinal movement without loss of fluid. Slider cap 95 is a further component of the assembly for the dynamic seal. The proximal end of injection tube 91 is bonded to coupling 94, which is connected to luer 93, which provides for input of fluid. A one-way valve, a pressure activated valve, or a luer-activated valve may be included to prevent blood escape when flushing is not needed.

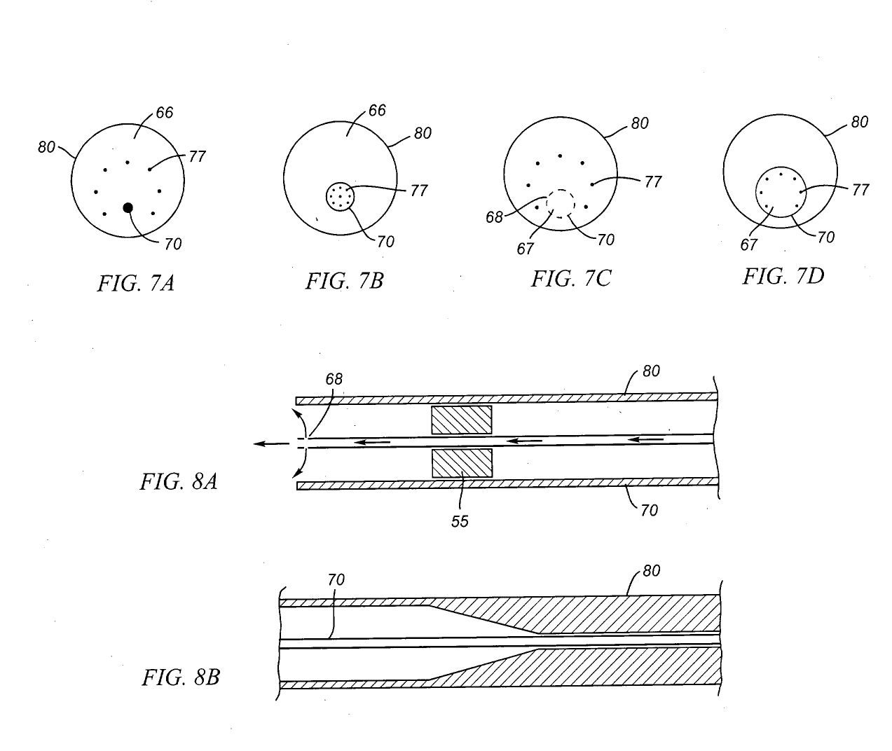

In this manner, fluid injected through luer 93 will pass through coupling 94, injection tube 91, and fill chamber 98. Fluid will then pass distally to port 88 and through the lumen of tubular member 80, thereby flushing the annuli between sliding components of the inner and outer assemblies. In cases where a tubular mandrel is used for flushing (e.g., FIGS. 7C and 7D), it may be desirable to dimension tubular member 71 and tubular member 81 (see FIG. 5C) so that a somewhat narrow annulus exists between these members when they are slideably assembled. Having a narrow annulus between these members will serve to maximize flushing distally to the annulus between the expansion frame and capture sheath, and minimize escape of saline proximally through the annulus between tubular members 71 and 81.

Various possibilities for the placement of temperature sensor wires and flushing lumens are shown in FIGS. 7A, 7B, 7C, and 7D, each of which is a cross-sectional view of FIG. 5C taken through section line 7-7. Temperature sensor wires 77 are attached distally to temperature sensors 13 (see FIG. 1A) and extend proximally beyond the useable length or working section of the thermography catheter and into a monitor that measures and records temperature readings taken from vulnerable plaque. As shown in FIG. 7A, annulus 66 between tubular member 80 of the outer assembly and mandrel 70 of the inner assembly may be used both for flushing and to carry temperature sensor wires 77. In the arrangement shown in FIG. 7B, mandrel 70 comprises a tubular structure. Tubular mandrel 70 carries temperature sensor wires 77 while annulus 66 provides flushing capabilities. In FIG. 7C, tubular mandrel 70 is equipped with flushing ports 68 near the distal end of mandrel 70. The lumen of mandrel 70 provides flushing capabilities while temperature sensor wires 77 are carried in the annulus between tubular member 80 and mandrel 70. Finally, FIG. 7D shows an arrangement wherein both temperature sensor wires 77 and flushing capabilities are provided through lumen 67 of mandrel 70.

Where flushing capabilities are provided through a tubular mandrel as shown in FIGS. 7C and 7D, it may be necessary to prevent escape of fluid proximally as shown in FIGS. 8A and 8B. Fluid for flushing travels distally through tubular mandrel 70 and flows through ports 68 into the annulus between tubular member 80 and mandrel 70. Toroidal seal 55 may be bonded to the inner surface of tubular member 80 to block proximal fluid flow. Alternatively, toroidal seal 55 may be bonded to the outer surface of mandrel 70 to block proximal fluid flow. In another alternative, tubular member 80 has a tapered inner lumen that fits tightly to mandrel 70, creating a very small annulus. In this manner, mandrel 70 remains slideable within tubular member 80, but fluid flow proximally through the annulus is prevented.

A lubricious coating may be provided on certain components to improve sliding of components. Teflon, parylene, or other materials may be used as the lubricious material. Mandrel 70 (FIG. 5A), tubular member 80 (FIG. 5B), and injection tube 91 (FIG. 6A) are among the components that will benefit from lubricious coating.

The working length of the thermography catheter will generally be between 50 and 200 centimeters, preferably approximately between 75 and 150 centimeters. The outer diameter of the thermography catheter shaft will generally be between 1 French and 8 French, preferably approximately between 1.5 French and 4 French. The diameter of the expansion frame when expanded will generally be between 1 and 10 mm, preferably approximately 2 and 5 mm for coronary artery applications. The foregoing ranges are set forth solely for the purpose of illustrating typical device dimensions. The actual dimensions of a device constructed according to the principles of the present invention may obviously vary outside of the listed ranges without departing from those basic principles.

Although the foregoing invention has, for the purposes of clarity and understanding, been described in some detail by way of illustration and example, it will be obvious that certain changes and modifications may be practiced that will still fall within the scope of the appended claims. For example, the devices and features depicted in any figure or embodiment can be used in any of the other depicted embodiments.

What is claimed is:

1. An intravascular thermography device comprising:
an elongate catheter having a proximal end, a distal end, a distal guidewire port in a distal region of the catheter, a proximal guidewire port at a location closer to the distal end of the catheter than the proximal end, and a lumen adapted to receive a guidewire and which extends between the proximal guidewire port and the distal guidewire port; and
an expansion frame attached to the catheter at a location distal to the proximal guidewire port, the expansion frame being operable between a contracted condition and an expanded condition, and having at least one temperature sensor.

2. The intravascular thermography device of claim 1, further comprising a capture sheath slideably disposed about the expansion frame and operable from the proximal end of the catheter to release the expansion frame when the capture sheath is removed from the expansion frame.

3. The intravascular thermography device of claim 2, wherein the capture sheath further comprises a slot in a region of the capture sheath near the distal end of the catheter, the slot communicating between a lumen of the capture sheath and an outside surface of the capture sheath, the slot being shaped to align with the proximal guidewire port of the catheter; and

4. The intravascular thermography device of claim 3, further comprising a registry mechanism operative to maintain circumferential alignment between the proximal guidewire port of the catheter and the slot of the capture sheath.

5. The intravascular thermography device of claim 1, wherein the temperature sensor is a thermocouple.

6. The intravascular thermography device of claim 1, further comprising a guidewire disposed within the lumen of the catheter and having a first portion of the guidewire extending out of the distal guidewire port, and having a second portion of the guidewire extending out of the proximal guidewire port and through the slot in the capture sheath.

7. The intravascular thermography device of claim 1, wherein the expansion frame comprises a plurality of struts that, upon activation, bow radially outward.

8. The intravascular thermography device of claim 1, wherein the expansion frame includes a plurality of temperature sensors.

9. An intravascular thermography device comprising:
an elongate catheter having a proximal end, a distal end, a distal guidewire port in a distal region of the catheter, a proximal guidewire port at a location closer to the distal end of the catheter than the proximal end, and a lumen adapted to receive a guidewire and which extends between the proximal guidewire port and the distal guidewire port;
an expansion frame attached to the catheter at a location distal to the proximal guidewire port, the expansion frame being operable between a contracted condition and an expanded condition, and having at least one temperature sensor; and
a capture sheath slideably disposed about the expansion frame and operable from the proximal end of the catheter to release the expansion frame when the capture sheath is removed from the expansion frame.

10. The intravascular thermography device of claim 9, wherein the capture sheath further comprises a slot in a region of the capture sheath near the distal end of the catheter, the slot communicating between a lumen of the capture sheath and an outside surface of the capture sheath, the slot being shaped to align with the proximal guidewire port of the catheter; and

11. The intravascular thermography device of claim 10, further comprising a registry mechanism operative to maintain circumferential alignment between the proximal guidewire port of the catheter and the slot of the capture sheath.

12. The intravascular thermography device of claim 9, wherein the capture sheath comprises a guiding catheter.

13. The intravascular thermography device of claim 9, wherein the capture sheath comprises an angiography catheter.

14. An intravascular thermography device comprising:
an elongate catheter having a proximal end, a distal end, a distal guidewire port in a distal region of the catheter, a proximal guidewire port at a location closer to the distal end of the catheter than the proximal end, and a lumen adapted to receive a guidewire and which extends between the proximal guidewire port and the distal guidewire port; and
an expandable member attached to the catheter at a location distal to the proximal guidewire port, the expansion frame being operable between a contracted condition and an expanded condition, and having at least one temperature sensor.

15. The intravascular thermography device of claim 14, wherein the expandable member is an expansion frame.

16. The intravascular thermography device of claim 14, further comprising a capture sheath slideably disposed about the expandable member and operable from the proximal end of the catheter to release the expandable member when the capture sheath is removed from the expandable member.

17. A method for detecting vulnerable plaque, comprising the steps of:
providing an elongate catheter having a proximal end, a distal end, a distal guidewire port in a distal region of the catheter, a proximal guidewire port at a location closer to the distal end of the catheter than the proximal end, and a lumen that extends between the proximal guidewire port and the distal guidewire port, the catheter further comprising an expansion frame in the distal region and having at least one temperature sensor;
positioning a guidewire across a region of interest within a target vessel;
inserting a proximal end of the guidewire into the distal guidewire port of the catheter, and through the proximal guidewire port of the catheter;
advancing the catheter along the guidewire until the expansion frame, is located within the region of interest;
deploying the expansion frame; and
operating the at least one temperature sensor to measure the temperature of an endoluminal surface of the vessel.

18. The method of claim 17, wherein the catheter further comprises a capture sheath slideably disposed about the expansion frame, and wherein the method further comprises the step of sliding the capture sheath relative to the expansion frame to release the expansion frame.

19. The method of claim 18, wherein the capture sheath further comprises a slot, the slot being shaped to align with the proximal guidewire port of the catheter, and wherein the guidewire passes through the slot.

20. The method of claim 17, wherein the step of positioning a guidewire across a region of interest within a target vessel is performed before the step of inserting a proximal end of the guidewire into the distal guidewire port of the catheter, and through the proximal guidewire port of the catheter.

21. The method of claim 17, wherein the step of inserting a proximal end of the guidewire into the distal guidewire port of the catheter, and through the proximal guidewire port of the catheter is performed before the step of positioning a guidewire across a region of interest within a target vessel.

22. A method for detecting vulnerable plaque, comprising the steps of:
providing an elongate catheter having a proximal end, a distal end, a distal guidewire port in a distal region of the catheter, a proximal guidewire port at a location closer to the distal end of the catheter than the proximal end, and a lumen that extends between the proximal guidewire port and the distal guidewire port, the catheter further comprising an expansion frame in the distal region and having at least one temperature sensor, the catheter further comprising a capture sheath slideably disposed about the expansion frame;
positioning a guidewire across a region of interest within a target vessel;
inserting a proximal end of the guidewire into the distal guidewire port of the catheter, and through the proximal guidewire port of the catheter;
advancing the catheter along the guidewire until the expansion frame is located within the region of interest;
sliding the capture sheath relative to the expansion frame to release the expansion frame
deploying the expansion frame; and
operating the at least one temperature sensor to measure the temperature of an endoluminal surface of the vessel.

23. The method of claim 22, wherein the capture sheath further comprises a slot, the slot being shaped to align with the proximal guidewire port of the catheter, and wherein the guidewire passes through the slot.

24. A method for detecting vulnerable plaque, comprising the steps of:
providing an elongate catheter having a proximal end, a distal end, a distal guidewire port in a distal region of the catheter, a proximal guidewire port at a location closer to the distal end of the catheter than the proximal end, and a lumen that extends between the proximal guidewire port and the distal guidewire port, the catheter further comprising an expandable member in the distal region and having at least one temperature sensor;
positioning a guidewire across a region of interest within a target vessel;
inserting a proximal end of the guidewire into the distal guidewire port of the catheter, and through the proximal guidewire port of the catheter;
advancing the catheter along the guidewire until the expandable member is located within the region of interest;
deploying the expandable member; and
operating the at least one temperature sensor to measure the temperature of an endoluminal surface of the vessel.

25. The method of claim 24, wherein the expandable member is an expansion frame.

26. The method of claim 24, wherein the catheter further comprises a capture sheath slideably disposed about the expandable member, and wherein the method further comprises the step of sliding the capture sheath relative to the expandable member to release the expandable member.

27. A thermography catheter comprising:
an outer assembly comprising an elongate tubular member having a proximal end, a distal end, and a lumen therebetween; and
an inner assembly inserted within the lumen of the outer assembly and slideable relative to the outer assembly, the inner assembly comprising a metallic elongate member having a proximal end and a distal end, an expandable member coupled to the distal end of the elongate member, the expandable member having at least one temperature sensor and being operable between a contracted condition and an expanded condition.
